(19) United States
(12) Patent Application Publication
(10) Pub. No.: US 2009/0232786 A1
(43) Pub. Date: Sep. 17, 2009

(54) HERBAL COMPOSITION FOR CANCER TREATMENT

(75) Inventor: Joanne HAMBROOK, Andrews (AU)

Correspondence Address: SUGHRUE MION, PLLC, 2100 PENNSYLVANIA AVENUE, N.W., SUITE 800, WASHINGTON, DC, 20037, US

(73) Assignee: Interhealth Biosciences PTY LTD., Burleigh (AU)

(21) Appl. No.: 12/401,658

(22) Filed: Mar. 11, 2009

Related U.S. Application Data
(63) Continuation of application No. 10/482,735, filed on Jul. 27, 2004, now abandoned.

(30) Foreign Application Priority Data
Jul. 5, 2001 (AU) PR 6006

(51) Int. Cl. A61K 35/66

(52) U.S. Cl. 424/93.51

(57) ABSTRACT

A therapeutic herbal composition comprising Saw Palmetto, Bromelain, Willow Herb, Grape Seed Complex, Wild Rosella, Liquorice, Passionfruit Seed and Selenium Yeast is claimed. The composition may be used as a general health promotant but is also useful as a treatment of cancer and inflammation. Prostate cancer and hyperplasia may be particularly beneficially addressed with the composition. The invention extends to a method of treatment using a composition as described.

This is a continuation application of U.S. Ser. No. 10/482,735 filed on Jan. 5, 2004, which is a national stage application under 35 U.S.C. § 371 of PCT/AU02/00903 filed Jul. 5, 2002, which claims priority from Australian Provisional Patent Application No. PR6006 filed Jul. 5, 2001; all of which are incorporated herein by reference.

### FIELD OF THE INVENTION

This invention relates to a method for using natural plant products in the promotion of health and treatment or prevention of disease. The invention particularly relates to the use of a herbal formula of naturally occurring products, derivatives thereof or synthetic equivalents for enhancing bodily, including prostatic health, and for the prevention or treatment of cancer, including prostatic cancer, and/or benign prostatic hyperplasia inflammation.

### BACKGROUND OF THE INVENTION

The prostate is a secretory gland located in the male genito-urinary tract. It primarily serves the purpose of providing secretory fluids for supporting sperm viability. The prostate however has a high incidence of age related pathology.

Prostatic cancer is a disease of global significance and is one of the major causes of death in adult males. Even when non-fatal, the disease can be intrusive, painful and disruptive to the lifestyle of a sufferer. Benign prostatic hyperplasia is also a common occurrence with significant side effects.

Traditional methods of treatment of prostatic neoplasia and even hypertrophy have involved the use of therapeutic agents such as anti-androgens including estrogens which have undesirable side effects. Additionally or alternatively, more radical means of treatment have involved the application of surgery, radiation therapy and chemotherapy. While providing good results in some patients, these therapies have a relatively high rate of unpleasant side effects and an unacceptably high rate of recurrence of the targeted disease and associated clinical signs.

Some attempts have been made to use natural remedies for the treatment of prostatic carcinoma. U.S. Pat. No. 5,665,393 is directed to a composition comprising material from the herbs: Panax pseudo-ginseng, Wall; Isatis indigotica, For; Ganoderma lucidum, Karst; Dendranthema morifolium, Tzvel; Glycyrrhzia glabra; Scutellaria baicalensis, Georgi; Rabdosia rubescens and Serenoa serrulata. This product is preferably provided in powdered concentrates from ethanolic extracts of dried plant material. The preparation has strong in vitro supporting evidence for the blocking of cell replication in the prostate cell line PC-3 (Darzynkiewicz et al. 2000). Studies were conducted in which PSA levels were significantly reduced using the formula disclosed and in some cases PSA levels dropped to below the limit of detection. However, significant side effects were reported in several of the studies (Small et al. 2000 and Oh et al. 2000). The side effects included loss of libido (100%) and potency (100%), gynecomastia/gynecodemia (100%), diarrhoea (33%) and leg cramps (64%), while some 6% of subjects experienced deep vein thrombosis (Darzynkiewicz et al. 2000). These side effects are, at least in part, due to the estrogenic activity which has long been a form of therapy for treatment of prostate pathologies. Serenoa serrulata has been well documented to contain phytoestrogens that lower endogenous estrogens in host animals as well as inhibit proliferation of benign and cancerous prostatic cells (Carilla et al., 1984; Iehlé et al., 1995; Delos et al., 1995).

It would be advantageous to produce a herbal tonic and remedy which provides an alternative to known treatments, particularly for prostatic cancer and benign prostate hyperplasia, and which minimises or has a reduced level of side effects during therapy.

Reference to any prior art in this specification is not and should not be taken as an acknowledgment or any form of suggestion that this prior art forms part of the common general knowledge in any country.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The bibliographic details of the publications referred to by author in this specification are collected at the end of the description.

The present invention is predicated in part on the identification and combination of a number of plants or parts thereof which are useful in the treatment or prophylaxis of cancer and/or inflammation, and in particular, prostatic cancer, and in the promotion of well being and health in a subject.

Accordingly, a first aspect of the present invention contemplates a herbal composition for the treatment or prophylaxis of a condition associated with neoplasia or a tendency to neoplasia, or for the promotion of health and well being, the composition comprising Saw palmetto, Bromelain, Willow herb, Grape seed complex, Wild rosella, Liquorice, Passionfruit seed and Selenium yeast.

In this context, Saw palmetto is Serenoa serrulata, Bromelain is or is derived from Ananas comosus. Willow herb includes Epilobium parviflorum. Grape seed complex includes Vitis vinifera. Wild rosella includes Hibiscus sabdariffa. Liquorice includes Glycyrrihza glabra L. Passionfruit seed includes or is derived from Passiflora edulis and preferably Passiflora edulis f. edulis. Selenium yeast is inactivated whole cell yeast product containing elevated levels of the essential trace element selenium in its natural food form.

In a preferred embodiment, the composition comprises ingredients in the following ranges:

| | | |
|---|---|---|
| Saw Palmetto | 5-90%; | |
| Bromelain | 1.5-21.5%; | |
| Willow Herb | 0.5-14.5%; | |
| Grape Seed Complex | 0.75-18.5%; | |
| Wild Rosella | 0.5-20%; | |
| Liquorice | 0.5-50%; | |
| Passionfruit Seed | 0.5-20%; | and |
| Selenium Yeast | 0.5-10%. | |

Reference in this specification to a plant either by way of its common name or species encompasses genetically modified plants. Genetically modified plants include transgenic plants or plants in which a trait has been removed or wherein an endogenous gene sequence has been down regulated, mutated or otherwise altered including the alteration or introduction of genetic material which exhibits a regulatory effect on a particular gene. Consequently, a plant which exhibits a characteristic not naturally present in a species referred to is nevertheless encompassed by the present invention and is included within the scope of the above mentioned term.

Extracts of the plants may be used to produce the composition. An extract may comprise sap or liquid or semi-liquid material exuded from or present in leaves, stems, flowers, seeds, roots, bark or between the bark and the stems. An extract may therefore comprise liquid or semi-liquid material located in fractions extracted from sap, leaves, stems, flowers, roots, bark or other plant material for example. Plant material may even be subject to physical manipulation to disrupt plant fibres and extra cellular matrix material and inter and intra tissue subject to extraction into a solvent including an aqueous environment. Alternatively, components of a plant may be mixed to form the composition of the invention. Those components may be dried and ground prior to mixture.

Reference herein to a plant includes various varieties, strains, lines, hybrids sub-species and variants or derivatives of the plant as well as its botanical or horticultural relatives. Furthermore, the present invention may be practised using a whole plant or parts thereof, using sap or seeds or any other reproductive material which may be used.

In a further aspect, the invention resides in a method of treating a subject, said method comprising the step of administering a therapeutically effective amount of a composition comprising Saw palmetto, Bromelain, Willow herb, Grape seed complex, Wild rosella, Liquorice, Passionfruit seed and Selenium yeast. Preferably the method is directed towards treatment of a mammal and most preferably a human. The method may be directed towards promoting well being or prostate health and/or preventing and/or treating prostatic cancer. The method may be directed to prevention or treatment of inflammation. The method may include the step of administering tablets/capsules to a subject. A treating physician may select any regime that provides a beneficial effect. However, a suitable regime may involve tablets/capsules administered once, twice or three times daily. A preferred regime is two to three tablets/capsules twice daily or 2000 mg to 4000 mg daily of combined product.

By “effective amount”, in the context of modulating an inflammatory response or treating or preventing a disease or condition, is meant the administration of that amount of composition to an individual in need thereof, either in a single dose or as part of a series, that is effective for that modulation, treatment or prevention. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

In still a further aspect, the present invention resides in a herbal composition when used in the manufacture of a medicament for the treatment or prevention of cancer or inflammation or otherwise promoting health the herbal composition comprising Saw palmetto, Bromelain, Willow herb, Grape seed complex, Wild rosella, Liquorice, Passionfruit seed and Selenium yeast.

In yet a further aspect, the invention contemplates use of a composition as described above when administered in a symptom ameliorating amount for a pathological condition and/or prophylaxis of said pathological condition.

### BRIEF DESCRIPTION OF DRAWINGS

FIG. 1 is a schematic representation of a cell divisional cycle.

FIG. 2 is a graphical representation of a control PC3 cell cycle assay.

FIG. 3 is a graphical representation of a PC3 cell cycle assay in the presence of paclitaxol.

FIG. 4 is a graphical representation of a PC3 cell cycle assay in the presence of a first level of a composition of the present invention.

FIG. 5 is a graphical representation of a PC3 cell cycle assay in the presence of a second higher level of the composition of FIG. 4 of the present invention.

FIG. 6 is a graphical representation of an LNCap cell cycle assay with a negative control.

FIG. 7 is a graphical representation of an LNCap cell cycle assay with a positive control.

FIG. 8 is a graphical representation of an LNCap cell cycle assay with a composition of the present invention.

FIG. 9 is a graphical representation of a HL60 cell cycle assay with a negative control.

FIG. 10 is a graphical representation of a HL60 cell cycle assay with a positive control.

FIG. 11 is a graphical representation of a HL60 cell cycle assay with a composition of the present invention.

FIG. 12 is a graphical representation of the cytotoxic effect of a composition of the present invention at different concentrations.

FIG. 13 is a graphical representation of percentage of cell growth inhibition plotted against the concentration of a composition of the present invention.

FIG. 14 is a graphical representation of the percentage of cell growth inhibition plotted against the concentration of the composition of FIG. 13.

FIG. 15 shows the results of competitive binding of estradiol.

FIG. 16 is a chemical profile of a composition of the present invention.

### DETAILED DESCRIPTION OF THE PREFERRED EMBODIMENT

The present invention is predicated in part on the identification of biologically useful properties of a herbal formula comprising naturally occurring herbs and plants or their botanical or horticultural relatives. The biologically useful properties include their use in the prophylaxis and/or treatment of cancer or neoplasia, extend to anti-inflammatory activity and also to general promotion of health, particularly prostate health.

The term “treatment” is used in its broadest sense and includes the inhibition of neoplasia or cancerous cell growth, the inhibition of growth or reduction in the size of a cancerous lesion as well as facilitating the amelioration of the effects of cancer and/or inflammation and promotion of good health in a subject.

The term “prophylaxis” is also used herein in its broadest sense to encompass a reduction in the risk of development of cancer, an inflammatory condition or other condition. The prophylactic administration of the composition may result in the composition becoming involved in the treatment of the pathological condition. Use of the terms “treatment” or “prophylaxis” is not to be taken as limiting the intended result which is to reduce the incidence of cancer and in particular prostatic cancer as well as or alternatively preventing or reducing inflammation or generally promoting health and well being or reducing or inhibiting benign prostatic hyperplasia.

Reference herein to a “subject” includes a human, primate, livestock animal (e.g. sheep, cow, horse, pig, goat, donkey), laboratory test animal (e.g. mouse, rat, guinea pig, hamster), companion animal (e.g. dog, cat) or avian species such as poultry birds (e.g. chicken, ducks, turkeys, geese) or game birds (e.g. ducks, pheasants).

The preferred subject is a human or primate or laboratory test animal.

The present composition is a balanced combination of six herbs, Selenium yeast and the natural enzyme Bromelain from pineapple. In the preferred embodiment, the composition provides a formulation that combines safety, efficacy and ease of use for a subject undergoing treatment with the invention.

Depending on the specific conditions being treated, the composition may be formulated and administered systemically or locally. Techniques for formulation and administration may be found in “Remington's Pharmaceutical Sciences,” Mack Publishing Co., Easton, Pa., latest edition. Suitable routes may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. For injection, the composition of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art. Intra-muscular and subcutaneous injection may be appropriate.

The composition may be formulated readily using pharmaceutically acceptable carriers well known in the art into dosages suitable for oral administration. Such carriers enable the compounds of the invention to be formulated in dosage forms such as tablets, pills, capsules, liquids, gels, syrups, slurries, suspensions and the like, for oral ingestion by a patient to be treated. These carriers may be selected from sugars, starches, cellulose and its derivatives, malt, gelatine, talc, calcium sulphate, vegetable oils, synthetic oils, polyols, alginic acid, phosphate buffered solutions, emulsifiers, isotonic saline, and pyrogen-free water.

Pharmaceutical compositions suitable for use in the present invention include compositions wherein the active ingredients are contained in an effective amount to achieve their intended purpose. The dose of agent administered to a patient should be sufficient to effect a beneficial response in the patient over time such as a reduction in the symptoms associated with the presence of a neoplasm or inflammation or to promote general well being and good health. The quantity of the agent(s) to be administered may depend on the subject to be treated inclusive of the age, sex, weight and general health condition thereof. In this regard, precise amounts of the composition for administration will depend on the judgement of the practitioner. In determining the effective amount of the chemical agent to be administered in the treatment or prophylaxis of a condition, the physician may evaluate tissue or fluid levels of the biological entity, and progression of the disorder. In any event, those of skill in the art may readily determine suitable dosages of the compositions of the invention.

Pharmaceutical formulations for parenteral administration include aqueous solutions of the herbal mixture in water-soluble form. Additionally, suspensions of the herbal mixture may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the composition or active compounds thereof with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinyl-pyrrolidone (PVP). If desired, disintegrating agents may be added, such as the cross-linked polyvinyl pyrrolidone, agar, or alginic acid or a salt thereof such as sodium alginate. Such compositions may be prepared by any of the methods of pharmacy but all methods include the step of bringing into association one or more chemical agents as described above with the carrier which constitutes one or more necessary ingredients. In general, the pharmaceutical compositions of the present invention may be manufactured in a manner that is itself known, e.g. by means of conventional mixing, dissolving, granulating, dragee-making, levigating, emulsifying, encapsulating, entrapping or lyophilising processes.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

Dosage forms of the invention may also include injecting or implanting controlled releasing devices designed specifically for this purpose or other forms of implants modified to act additionally in this fashion. Controlled release of an agent of the invention may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, controlled release may be effected by using other polymer matrices, liposomes and/or microspheres.

The herbal components of the invention may be provided as salts with pharmaceutically compatible counterions. Pharmaceutically compatible salts may be formed with many acids, including but not limited to hydrochloric, sulphuric, acetic, lactic, tartaric, malic, succinic, etc. Salts tend to be more soluble in aqueous or other protonic solvents that are the corresponding free base forms.

For any composition used in the method of the invention, the therapeutically effective dose can be estimated initially from cell culture assays. For example, a dose can be formulated in animal models to achieve a circulating concentration range that includes the IC₅₀ as determined in cell culture. Such information can be used to more accurately determine useful doses in humans.

Toxicity and therapeutic efficacy of such chemical agents can be determined by standard pharmaceutical procedures in cell cultures or experimental animals, e.g. for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferred. The data obtained from these cell culture assays and animal studies can be used in formulating a range of dosages for use in humans. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition (see for example Fingl et al., 1975, in “The Pharmacological Basis of Therapeutics”, Ch. 1 p 1).

Dosage amount and interval may be adjusted individually to provide plasma levels of the active agents which are sufficient to maintain symptom-ameliorating effects. Usual patient dosages for systemic administration range from 1-5000 mg/day, commonly from 1-3000 mg/day. Stated in terms of patient body weight, usual dosages range from 0.02-80 mg/kg/day, commonly from 0.02-50 mg/kg/day, typically from 0.2-50 mg/kg/day. Stated in terms of patient body surface areas, usual dosages range from 0.5-2500 mg/m²/day, commonly from 0.5-1500 mg/m²/day, typically from 5-1500 mg/m²/day.

Alternatively, one may administer the compound in a local rather than systemic manner, for example, via injection of the compound directly into a tissue, often in a depot or sustained release formulation. Furthermore, one may administer the drug in a targeted drug delivery system, for example, in a liposome coated with tissue-specific antibody. The liposomes will be targeted to and taken up selectively by the tissue. In cases of local administration or selective uptake, the effective local concentration of the agent may not be related to plasma concentration.

The herbal composition of the invention can also be delivered topically. For topical administration, a composition containing between 0.001-5% or more composition is generally suitable. Regions for topical administration include the skin surface and also mucous membrane tissues of the vagina, rectum, nose, mouth, and throat. Compositions for topical administration via the skin and mucous membranes should not give rise to signs of irritation, such as swelling or redness.

The topical composition may include a pharmaceutically acceptable carrier adapted for topical administration. Thus, the composition may take the form of a suspension, solution, ointment, lotion, sexual lubricant, cream, foam, aerosol, spray, suppository, implant, inhalant, tablet, capsule, dry powder, syrup, balm or lozenge, for example. Methods for preparing such compositions are well known in the pharmaceutical industry.

In one embodiment, the composition may be administered topically to a subject, e.g. by the direct laying on or spreading of the composition on the epidermal or epithelial tissue of the subject, or transdermally via a “patch”. Such compositions include, for example, lotions, creams, solutions, gels and solids. Suitable carriers for topical administration preferably remain in place on the skin as a continuous film, and resist being removed by perspiration or immersion in water. Generally, the carrier is organic in nature and capable of having dispersed or dissolved therein a chemical agent of the invention. The carrier may include pharmaceutically-acceptable emolients, emulsifiers, thickening agents, solvents and the like.

A preferred formulation of the composition of the invention is prepared to provide a balanced combination of Saw palmetto (Serenoa serrulata), the natural enzyme bromelain from pineapple and six herbs including Willow herb (Epilobium parviflorum), Grape seed complex (Vitis vinifera), Wild rosella (Hibiscus sabdariffa), Liquorice (Glycyrrihza glabra L.), Passionfruit seed (Passiflora edulis f. edulis) and Selenium yeast. The components are preferably utilised in the dried powder form.

A composition of the herbal formula is preferably presented as a tablet/capsule which may have approximately 500˜800 mg of active ingredients per tablet/capsule. Examples of typical formulation of active ingredients may be produced with percentage active ingredient on a weight basis as set out in Table 1:

TABLE 1
Twenty-five formulas of representative compositions of the present invention
Summaries (VALUES IN PERCENTAGES)

| | SAW | | WILLOW | GRAPE SEED | | | PASSION | SELENIUM |
|---|---|---|---|---|---|---|---|---|
| | PALMETTO | BROMELAIN | HERB | COMPLEX | WILD ROSELLA | LIQUORICE | FRUIT | YEAST |
| 1 | 90.00 | 4.50 | 2.25 | .75 | .50 | .50 | .50 | 1.00 |
| 2 | 85.00 | 1.50 | 4.50 | 4.50 | .50 | 2.00 | 1.00 | 1.00 |
| 3 | 80.00 | 8.50 | .50 | 1.50 | 2.50 | 2.50 | .50 | 4.00 |
| 4 | 75.00 | 8.50 | 1.00 | 1.00 | 1.00 | .50 | 3.00 | 10.00 |
| 5 | 28.20 | 10.60 | 2.80 | 2.80 | 1.40 | 49.30 | 1.40 | 3.50 |
| 6 | 38.80 | 10.60 | 2.80 | 2.80 | 2.20 | 37.20 | 1.60 | 4.00 |
| 7 | 48.50 | 10.60 | 3.80 | 2.80 | 2.10 | 25.00 | 2.20 | 5.00 |
| 8 | 20.20 | 12.60 | 5.80 | 13.80 | 12.80 | 23.80 | 4.50 | 6.50 |
| 9 | 15.50 | 15.00 | 8.80 | 10.60 | 20.00 | 15.00 | 12.60 | 2.50 |
| 10 | 10.50 | 15.00 | 9.50 | 16.50 | 12.50 | 18.50 | 15.00 | 2.50 |
| 11 | 5.00 | 15.00 | 9.50 | 18.50 | 14.50 | 19.50 | 16.00 | 2.00 |
| 12 | 10.00 | 12.00 | 5.50 | 16.50 | 15.50 | 15.50 | 20.00 | 5.00 |
| 13 | 55.00 | 12.50 | 6.50 | 5.20 | 7.80 | 5.20 | 5.00 | 2.80 |
| 14 | 41.00 | 13.00 | 7.50 | 4.50 | 12.50 | 9.50 | 6.50 | 5.50 |
| 15 | 35.50 | 18.50 | 13.50 | 4.00 | 12.00 | 8.50 | 5.50 | 2.50 |
| 16 | 30.50 | 20.50 | 10.00 | 5.50 | 5.50 | 12.50 | 10.50 | 5.00 |
| 17 | 25.00 | 21.50 | 14.50 | 5.50 | 6.50 | 11.50 | 11.50 | 4.00 |
| 18 | 65.00 | 10.00 | 5.00 | 5.00 | 5.00 | 3.00 | 4.00 | 3.00 |
| 19 | 61.50 | 5.50 | 6.50 | 10.50 | 3.20 | 4.50 | 3.50 | 4.80 |
| 20 | 45.50 | 18.50 | 5.50 | 11.50 | 6.50 | 7.50 | 4.50 | .50 |
| 21 | 32.00 | 13.50 | 9.50 | 16.50 | 10.50 | 8.50 | 4.50 | 5.00 |
| 22 | 70.00 | 5.00 | 5.00 | 5.00 | 5.00 | 3.50 | 3.00 | 3.50 |
| 23 | 50.00 | 8.00 | 2.50 | 2.50 | 5.50 | 25.50 | 2.50 | 3.50 |
| 24 | 42.50 | 10.50 | 4.50 | 4.50 | 2.50 | 30.50 | 3.00 | 2.00 |
| 25 | 72.00 | 9.50 | 3.00 | 4.00 | 3.50 | 5.00 | 1.50 | 1.50 |

It is important to note that the above ratios and percentages are approximate only. The components of the herbal formula provided in Table 1 are natural products. As such they are prone to considerable variation in strength due to a wide range of influences including climatic, geographical and genetic conditions. The concentration of the ingredients in the therapeutic composition of the invention may be beneficially adjusted to accommodate variation in activity in particular ingredients. The preferred range of components are therefore Saw Palmetto 5-90%; Bromelain 1.5-21.5%, Willow Herb 0.5-14.5%, Grape Seed Complex 0.75-18.5%; Wild Rosella 0.5-20%; Liquorice 0.5-50%; Passionfruit Seed 0.5-20% and Selenium yeast 0.5-10% calculated on a weight basis.

A tablet may be formed with additional components which may include excipients and binders as used in the pharmaceutical industry.

While the following discussion is directed to anti-cancer therapy, it should be understood this is not limiting. The composition may be used as a general tonic and promoter of good health, in particular, prostate gland health. Further the application in cancer prophylaxis and treatment may extend beyond prostatic neoplasia and include a range of tumours including sarcomas and carcinomas of other organs and sites.

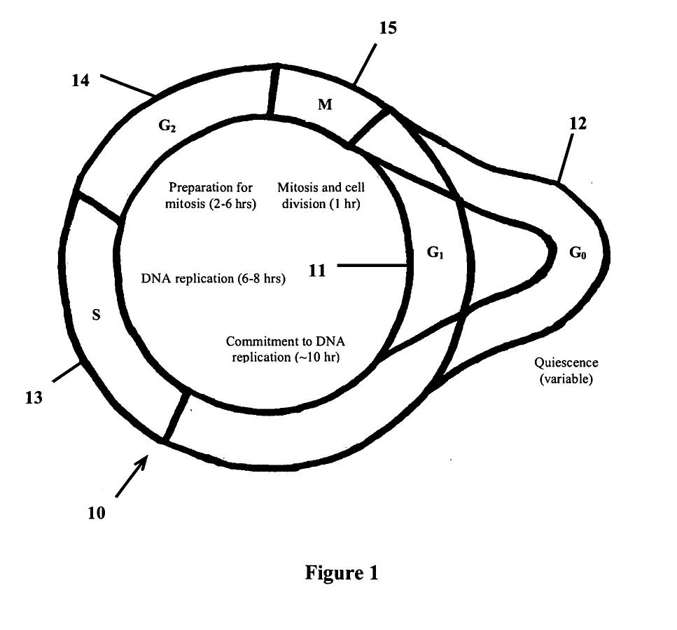

The present composition has distinct advantages over some prior art remedies and therapies both in terms of its efficacy and decreased level of side effects. Referring to FIG. 1, there is seen a schematic representation of a cell cycle 10 (adapted from Voet & Pratt 1999). The process by which cells divide and DNA is replicated is a complicated process in eukaryotic cells and involves a number of different phases. Cancerous cells are dividing cells and so will exhibit a well defined cell cycle which is separated into several distinct phases. It starts at the G₁ phase 11 or the first gap phase. If the cells are permanently arrested in G₁, as in non-dividing cells, it is then called G₀ 12. At G₁ the cell contains two copies of each chromosome. As the cell cycle progresses from G₁, it enters the synthesis or S phase 13 and during this phase DNA is replicated. When this replication is finished, the cell enters the G₂ phase or the second gap phase 14. At the end of the G₂ phase the cell is ready to enter the M phase or mitotic phase 15 during which the cell divides. Using cell cycle analysis it is possible to break down the events into the major phases such as G₀-G₁, G₂-M and S. Many drugs used in the fight against cancer affect the cell cycle at these points.

### Example 1

Cell cycle analysis was carried out using tablets/capsules of the present invention. The formulation was tested as a single tablet/capsule concentration in triplicate and as a two capsule concentration also in triplicate. The effects on the cell cycles at various concentrations were therefore comparable.

Tablets/capsules were weighed, crushed and extracted with 10 ml of methanol. The extracts were sonicated for 40 mins and then spun down at 4000 rpm. The supernatant was removed and 100 μL of this extract was used in the cell cycle analysis. A further aliquot of this extract was subjected to HPLC-MS analysis as described in Example 3 below. Cells were split into 16×25 cm² flasks at a low concentration and allowed to grow to 65-70% confluence (at the time of addition the cells were in log phase). Flasks were incubated for 24 hours and control flasks of control media (no additions), an ethanol (100 μL EtOH), a control methanol (100 μL MeOH) and paclitaxol (10 μL of 0.01 mg/mL=10 ng/mL) were also run.

Flow cytometry was performed on the fixed cells after washing in PBS and stained using propidium iodide (2% PI in 0.1% Triton X-100 containing 2 mg/mL Rnase A). A Becton Dickson FACSCaliber was used to assay the cells.

Cell cycle analysis using flow cytometry of propidium iodide stained cells showed the tablets affect the cell cycle of PC-3 cell lines by arresting the cell cycle in G₂-M. Even a low dose of the invention preferentially arrested the cells in G₂-M. While the cell line is a prostate cancer cell line it is possible to apply the same therapy to other forms of cancer cell lines and therefore to other cancers.

The following table sets out the results of the test wherein formulations of the present invention are identified as Herb Formula:

TABLE 2
Percentage Changes in G₀-G₁ and G₂-M

| | G₀-G₁ | G₂-M |
|---|---|---|
| Control | 43.74 | 25.43 |
| Herb Formula-1-1 | 14.08 | 75.35 |
| Herb Formula -1-2 | 7.88 | 74.92 |
| Herb Formula -1-3 | 18.08 | 55.39 |
| Mean ± SD | 13.35 ± 5.14 | 68.55 ± 11.40 |
| Herb Formula -2-1 | 4.45 | 82.12 |
| Herb Formula -2-2 | 20.91 | 63.88 |
| Herb Formula -2-3 | 8.76 | 74.53 |
| Mean ± SD | 11.37 ± 8.54 | 73.51 ± 9.16 |

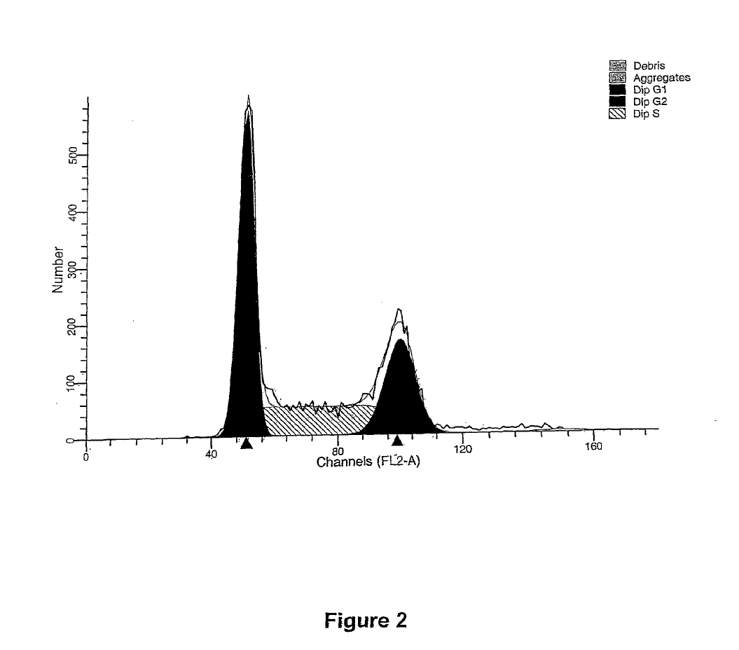

It is apparent in Table 2 that the result for the control shows that only 44% of the cells are in the G₀-G₁ phase while 25% are in the G₂-M phase. With increasing dosage of the tablet/capsule of the invention, the mean percentage of cells in the G₀-G₁ was in the range of 13.35 to 11.37%. Meanwhile the percentage of cells in the G₂-M phase had risen to be between on average 68.55-73.51%. This indicates a locking or arresting of cells in the G₂-M stage and subsequent prevention of mitotic and cellular growth. FIG. 2 is a histogram of the results of flow cytometry performed on the control. In this result 43.74% of the cells were in the G₀-G₁ phase at 50.80, 25.43% were in the G₂-M phase at 99.88. 30.83% of the cells were in the S phase.

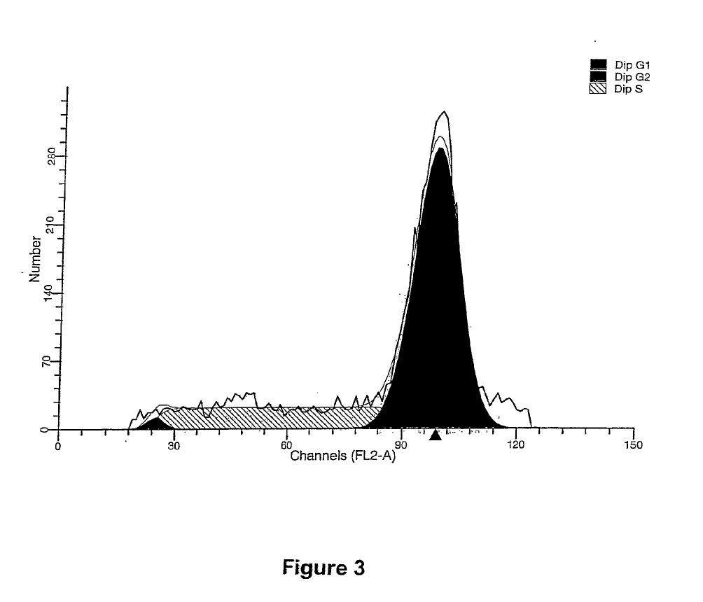

FIG. 3 shows the results when using Paclitaxol, a well known chemotherapeutic agent for use against cancer. In this result, 1.16% of the cells are in the G₀-G₁ phase at 25.06, 71.89% of the cells in the G₂-M phase at 98.40. This highlights the fact that the cells are arrested at the G₂-M phase with this drug. 26.95% were in the S phase.

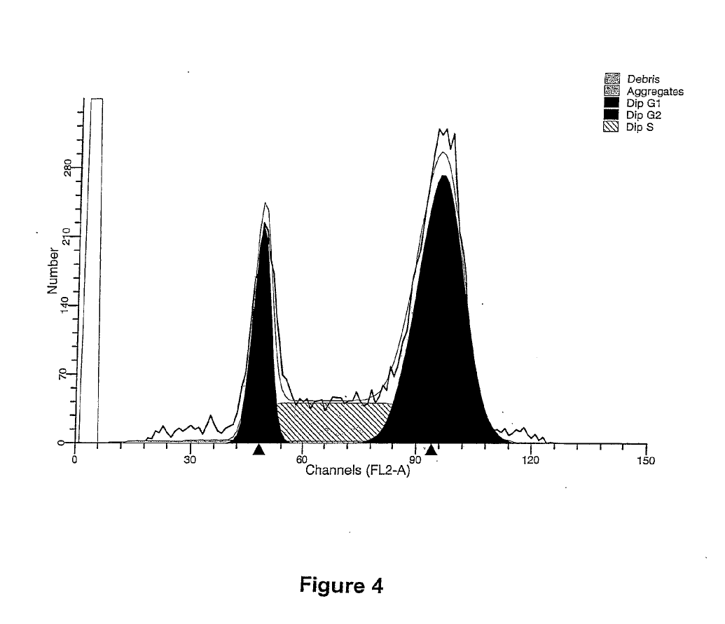

FIG. 4 shows the results of using one tablet/capsule of the present invention. 18.08% of the cells were in the G₀-G₁ phase at 48.12. 55.39% of the cells were in the G₂-M phase at 95.52 while 26.53% were in the S phase.

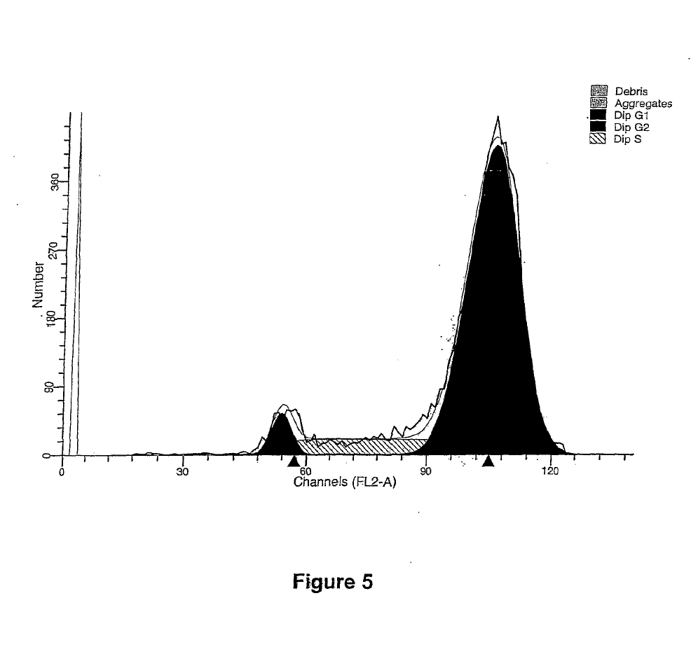

FIG. 5 shows the results of using two tablets/capsules of the present invention. The result here is even more marked in that only 4.45% of the cells were in the G₀-G₁ phase at 53.86. Meanwhile 82.12% of the cells were in the G₂-M phase at 105.22 and 13.43% of the cells were in the S phase.

These results highlight the therapeutic effectiveness of the present invention against replication of prostatic carcinoma cells lines. Tests have also shown that in clinical use, this product may produce lowering of prostatic specific antigen (PSA) which is a well known indicator of prostatic hyperplasia and prostatic carcinoma.

The present invention has also been shown to have an anti-inflammatory effect and may be of use in inflammatory conditions such as arthritis and autoimmune diseases. The invention may also be useful against secondary inflammation produced as a result of primary infectious or traumatic processes.

### Example 2

A anti-cancer activity assay was conducted with the results presented in Table 3 wherein the herb formula represents a combination according to the present invention. LNCap is an androgen-dependent prostate cancer cell line.

From cell cycle analysis using flow cytometry of promidium iodide stained cells 3.2 and 1.6 mg/mL concentrations of the herb formula affect the cell cycle of LNCap cell lines by arresting the cell cycle in G₂-M. The herb formula showed dose-dependent effects on LNCap cell lines assay.

TABLE 3
Herb Formula bioactivity using LNCap cell cycle assay

| | Concentration | | |
|---|---|---|---|
| Treatment | (mg/10 mL) | G₀-G₁ | G₂-M |
| Negative control | | 80.79 | 5.31 |
| Herb Formula | 0.4 | 62.72 | 10.83 |
| | 0.8 | 72.23 | 9.91 |
| | 1.6 | 10.88 | 79.69 |
| | 3.2 | 10.73 | 77.23 |

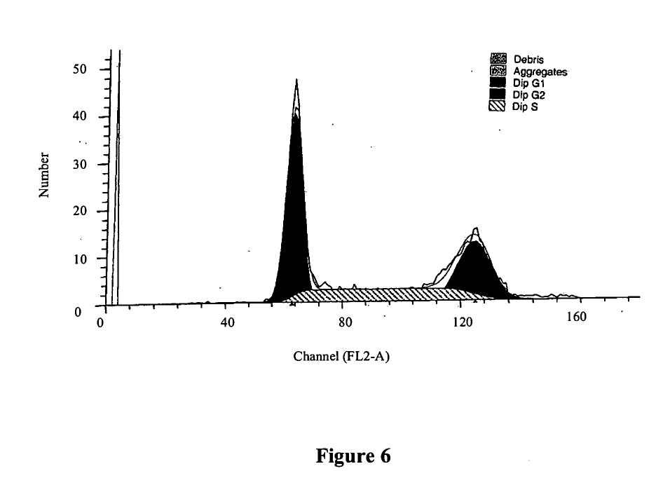

FIG. 6 is a graphical representation of the DNA histogram of a LNCap cell line with a negative control.

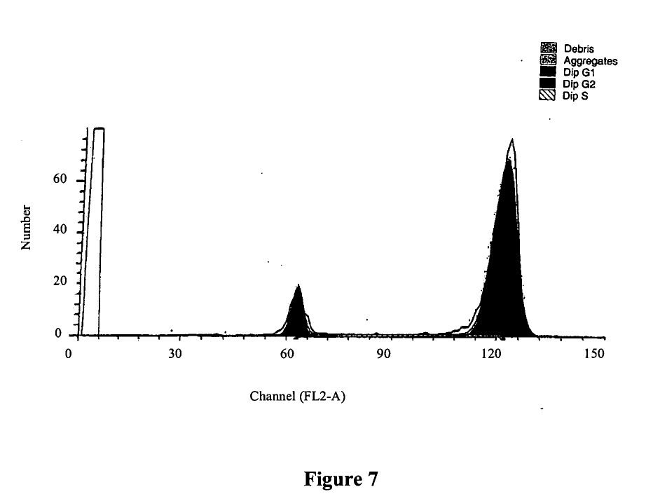

FIG. 7 is a graphical representation of an LNCap cell cycle assay with a positive control in the form of Paclitaxol showing arresting of cells in G2-M.

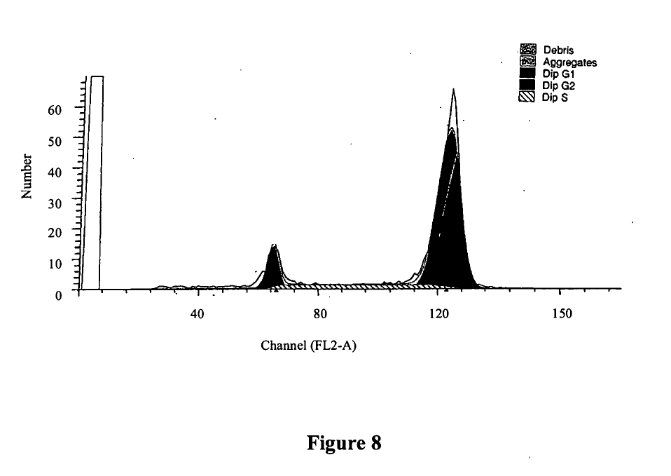

FIG. 8 shows the results of a combination of the present invention, the herb formula composition in contact with an LNCap cell cycle assay. As noted, the herb formula shows a dose dependent effect on LNCap cell lines and arrests the cells in the G₂-M stage.

### Example 3

The bioactivity of the herb formula was assessed using HL60 cell lines. HL60 is a human promyelocytic leukemia cell line.

From cell cycle analysis using flow cytometry of promidium iodide stained cells 3.2 and 1.6 mg/mL concentrations of the herb formula affect the cell cycle of HL60 cell lines by arresting the cell cycle in G₂-M. Herb Formula showed dose-dependent effects on HL60 cell lines assay as shown in Table 4.

TABLE 4
The herb formula bioactivity using HL60 cell cycle assay

| | Concentration | | |
|---|---|---|---|
| Treatment | (mg/10 mL) | G₀-G₁ | G₂-M |
| Negative control | | 34.79 | 12.77 |
| Herb Formula | 0.4 | 31.65 | 13.74 |
| | 0.8 | 32.44 | 12.07 |
| | 1.6 | 28.65 | 21.89 |
| | 3.2 | 12.34 | 52.68 |

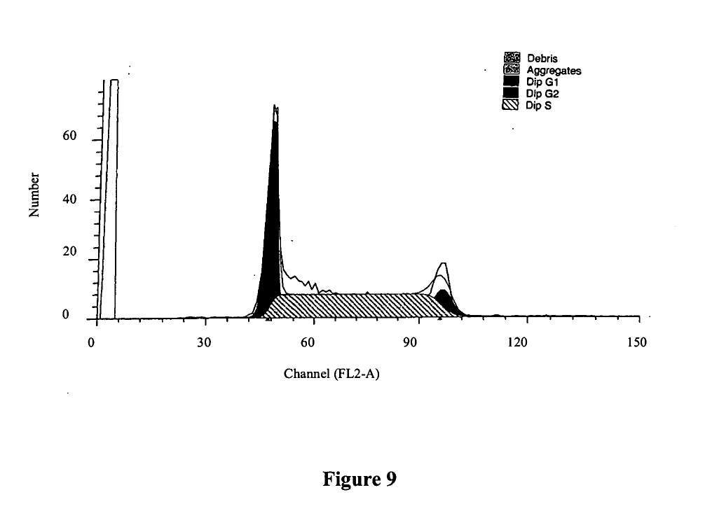

FIG. 9 shows a histogram of cell cycle analysis for an HL60 cell line with a negative control.

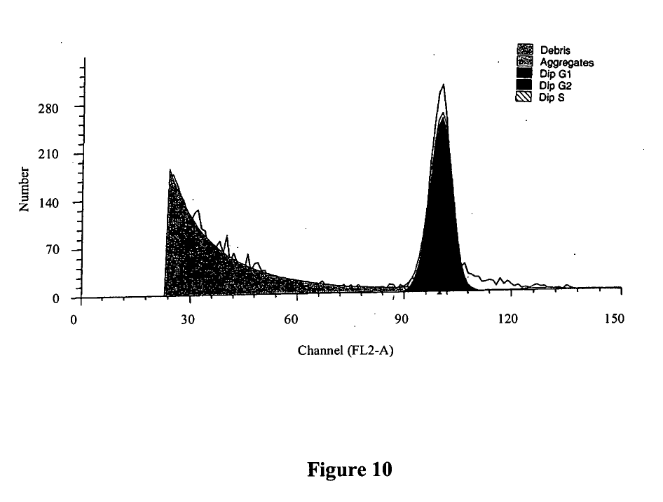

FIG. 10 shows a graphical representation of the DNA histogram for an HL60 cell line with a positive control in the form of Paclitaxol. The effect is to arrest the cells in the G₂-M stage.

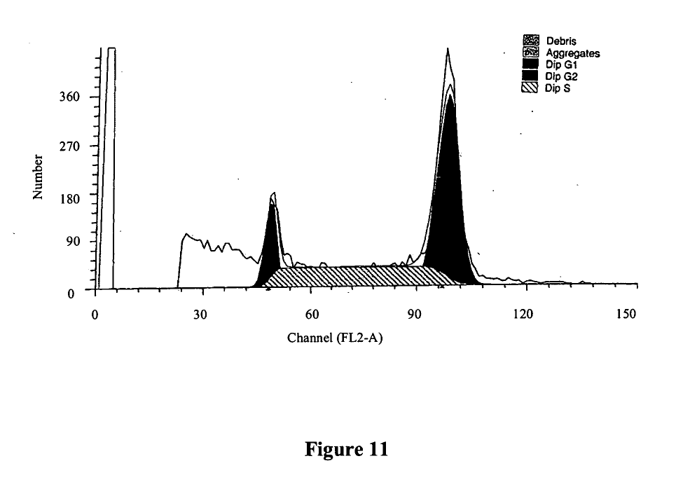

FIG. 11 shows a graphical representation of the DNA histogram for an HL60 cell line with the herb formula. The mixture displays chemotherapeutic benefit by arresting the cells in the G₂-M stage.

### Example 4

As noted, the herbal formula may be used as an anti-inflammatory agent. This capacity was investigated with a bioactivity assay

This assay was run to determine the effectiveness of the herbal formula to inhibit Prostaglandin E₂ (PGE₂) production in a mouse 3T3 fibroblast cell line.

The anti-inflammatory assay measures the level of PGE₂ in stimulated mouse fibroblast 3T3 cell line after treatment with the herbal formula. The use of a stimulated cell line rather than using isolated enzyme systems gives more useful data.

PGE₂ is formed in a variety of cells from PGH₂, which itself is synthesized from arachiodonic acid by the enzyme prostaglandin synthetase. The assay measures secreted levels of PGE₂ from the supernatant of the fibroblast cells.

The herbal formula had significant dose dependent inhibition of secreted PGE₂ from the mouse fibroblast cell line equivalent to or better than aspirin under the test conditions.

TABLE 5
Percent inhibition of secreted PGE₂ from
3T3 cells exposed to the herb formula

| | | Concentration | |
|---|---|---|---|
| Sample | Dilution | (μg/mL) | % Control |
| Aspirin | 500 | 500 μM | 14.2 |
| Herb Formula | 500 | 1000 | 15.8 |
| Herb Formula | 500 | 100 | 30.0 |

### Example 5

Safety and toxicity of the herbal formula were investigated using a cytotoxicity assay.

Cells were split into 10 cm² tissue culture tubes at a low concentration and allowed to grow to confluent. Media was DMEM with 10% horse sera (GIBCO) with glutamine and pen/strep added tubes placed in a 10% CO₂ incubator. Cells were then transferred to tissue culture plates in 99 μL media. Samples for testing were then added, 1 μL into each well ranging from 1 mg/mL to 0.001 mg/mL. The assay is based on the presence of ATP in living cells and is measured using luminescence readings on the Wallac Microbeta.

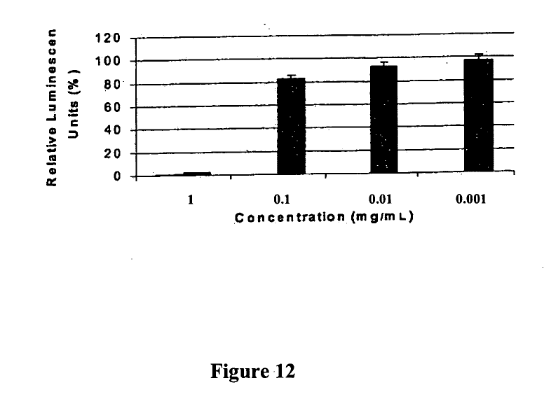

The herb formula only had slight cytotoxic affect at the 0.1 mg/mL doses and almost no affect at the 0.01 and 0.001 mg/mL dose as shown in FIG. 12.

### Example 6

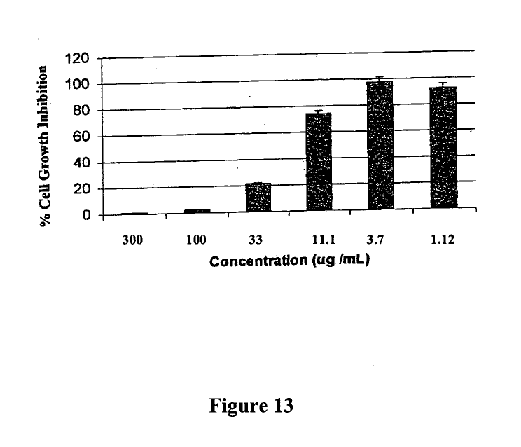

The toxicity of the herbal formula was also investigated using HL60 cell lines. The herbal formula had cytotoxic affect at the 100 μg/mL doses, had a slight cytotoxic effect at the 33 μg/mL dose and had no affect at the 3.7 and 1.12 μg/mL dose. The results are shown in FIG. 13 which is a chart of percentage cell growth inhibition against concentration of Herbal Formula.

### Example 7

The toxicity of the herbal formula was also investigated using HepG2 cell lines. Unlike the other cell lines, the herbal formula had slight cytotoxic affect at the 300 μg/mL doses and had no affect at the 100, 33, 11.1, 3.7 and 1.12 μg/mL dose.

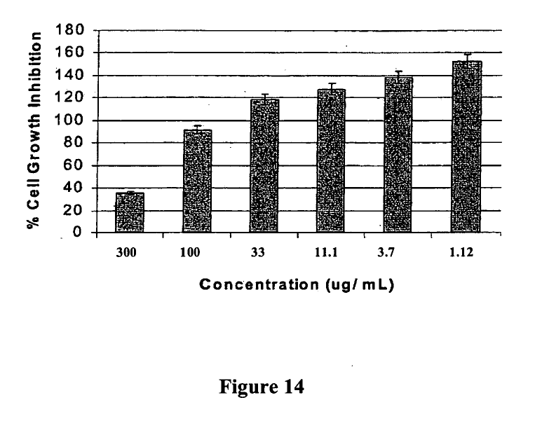

The results are shown in FIG. 14 which is a chart of percentage cell growth inhibition against concentration of the herb formula.

### Example 8

The estrogen binding capacity of the herbal formula was investigated by assay. This assay was developed and run to determine if the herbal extracts that constitute the herbal formula have any binding activity towards the estrogen (E₂) binding site on MCF-7 cell lines. The MCF-7 is a breast cancer cell line and was chosen because of the ability to express E₂ binding sites.

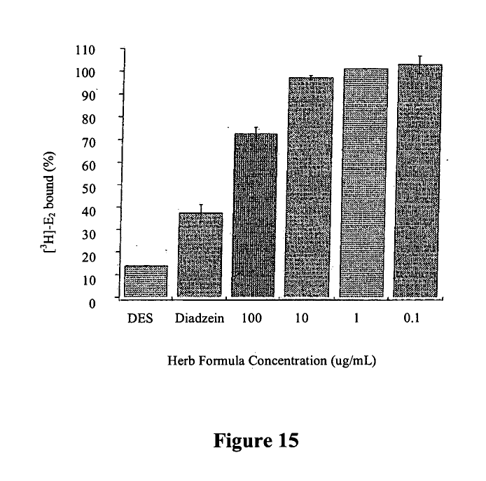

³H 17-β-estradiol (E₂) was used at a final concentration in the wells of 1 nM in media. Samples were incubated for 3 hours after which the wells were aspirated and washed 3 times with warm PBS. Cell bound radioactivity was extracted with 300 μL EtOH which was added to each well and left to lyse for 20 min at room temperature. An aliquot was removed for counting, transferred to a counting plate and scintillant added and the plate counted in a Perkin Elmer MicroBeta. Diethylstilbestrol (DES) (100 μM) and diadzein (197 μM) were used as positive binding controls. Cold 17-β-estradiol (non-radioactive) (0.051-12.5 nM) was used as a competitor for the E₂ binding sites. A competitive binding of ³H 17-β-estradiol against cold 17-β-estradiol was demonstrated by this assay, the results of which are shown in FIG. 15. As expected both DES (100 μM) and diadzein (197 μM) bind to the E₂ receptor displacing over 85% and 60% of the ³H E₂, respectively.

Herb Formula however has less than 30% inhibition of ³H 17-β-estradiol binding to E₂ receptors at the highest concentration tested (100 μg/mL). The other concentrations of the herb formula had little or no effect on the inhibition of ³H 17-β-estradiol binding. From these results it can not be predicted with certainty whether or not the composition will have estrogenic activity in whole animals, it can however be inferred that since there is little or no binding to the E₂ receptors (at the concentrations tested) it would seem unlikely that estrogenic activity could occur. This would also suggest that the herb formula may well be free of associated disturbing side-affects (breast tenderness, loss of libido etc).

### Example 9

Toxicity of the present invention was assessed using P₃₈₈ cell line toxicity assay. Cells were split into 10 cm² tissue culture tubes at a low concentration and allowed to grow until confluent. The media used was DMEM with 10% horse sera (GIBCO) with glutamine and PEN-STREP added tubes placed in a 10% CO₂ incubator. Cells were then transferred to tissue culture plates in 99 μL media. Samples for testing were then added (1 μL into each well ranging from 1 mg/ml to 0.001 mg/ml). The assay was based on the presence of ATP in living cells and was measured using luminescence readings on the Wallac Microbeta. Tablets of the present invention showed a level of cytotoxity at the higher dose of 1 mg/ml. However, it had only slight cytotoxic effect at the 0.1 mg/ml dose and almost no effect at 0.01 and 0.001 mg/ml doses. This lack of cytotoxicity shows a particular advantage for the present invention as in minimises risk to healthy tissues.

Although any suitable route of administration may be employed for providing the patient with an effective dosage of the composition according to the present invention, oral administration is preferred. Suitable alternative routes include, for example, oral, rectal, parenteral, intravenous, topical, transdermal, subcutaneous, intra muscular and similar forms of administration. Suitable dosage forms include tablets, troches, dispersions, suspensions, solutions, capsules, patches, suppositories and the like although oral dosage forms are preferred.

### Example 10

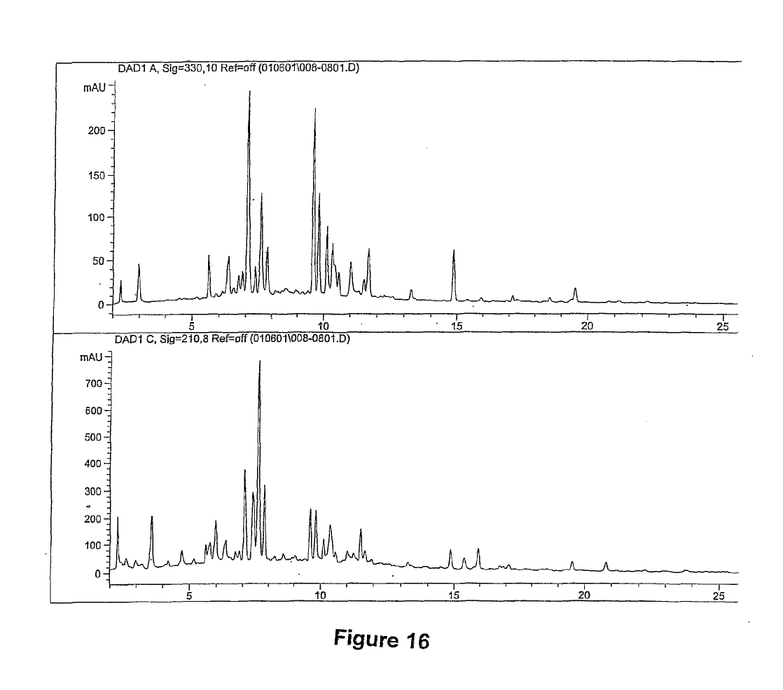

The chemical profile of the extract prepared as outlined in Example 1 was obtained using an Agilent 1100 HPLC coupled to a diode array detector and mass spectrometer. Separation was achieved on a 5μ C₁₈ column (15 cm×4.6 cm; Phenomonex) using a linear gradient from water (95%) to acetonitrile (95%) over forty minutes at a flow rate of 1 mL/min. A typical fingerprint of this invention should display the characteristic peak profile displayed in FIG. 16. The chromatograms in this example were generated at 330 nm and 210 nm from the diode array data.

### Example 11

A white male, age 74, presented for a biopsy and bone scan which revealed prostate cancer without bony metastases in 1998. Serum PSA was measured to be 300. This patient then started hormone therapy including an oral administration of 100 mg of Androcur (Cyproterone Acetate) 3 times per day, and a subcutaneous injection of 3.6 mg of Zolodex (Goserelin Acetate Implant) once per three months. The prostate has been monitored since with serum PSA levels, varying between “28-62”.

In May 2001, the PSA level was measured to be 41. At that time, the patient stopped taking Androcur and Zolodex, and started to take Casodex 50 mg once daily. The treatment with Casodex continued for three months and follow-up blood tests indicated a serum PSA of 5.7.

From August 2001, this patient started to combine Casodex with an oral administration of the herbal formula of the present invention. The intake dosage of the herbal formula was 2070 mg per day. This combination treatment continued for two months. During the two months, the patient's energy and well-being were enhanced, and no adverse effects were reported.

From October 2001, the dosage of this herb formula was increased to 2760 mg per day. Following PSA level measurement was 2.3 in November 2001. One month later (December 2001) blood tests showed a constant PSA reading of 2.3.

### Example 12

The patient in this case study was a 63-year-old white male. A biopsy was performed in 1996, and the pathology report had indicated Benign Prostatic Hyperplasia. The PSA was measured to be 21.

The prostate has been monitored approximately every 6 months. As the PSA reading was between “13-17” on occasions, when readings were high, antibiotics were prescribed for prostatitis. The PSA was measured to be 18.7 at August of 2001.

Biopsy was performed again in November of 2001 and nine samples were taken in the biopsy and cancer was detected in one sample only. The urologist suggested a “watchful waiting” position should be adopted and then recommended a discussion with an Oncologist. The patient did contact an Oncologist, and radiation therapy was suggested.

The patient did not take radiotherapy, and started a therapy by oral administration of the herbal formula of the present invention. The intake dosage of herbal formula was 2760 mg per day. This combination treatment continued for one and a half months and a follow-up serum PSA of 18.1 was reported. During this period, the patient's energy and well-being were enhanced. No adverse effects were reported.

### Example 13

A white male, age 58, presented a PSA level of 8.6. One week later, this patient started to take therapy by oral administration of the herbal formula of the present invention. The intake dosage of the herbal formula was 1380 mg per day. This patient did not take any hormone therapy.

This herbal formula continued for one month, and a follow-up serum PSA of 3.7 was reported. During this period, the patient's energy and well-being were enhanced and no adverse effects were reported. This patient increased the dosage to 2760 mg per day. Four months later, the follow-up serum PSA level was reported to be 2.1.

### Example 14

A white male, age 69, presented for a PSA level of 4.4 in July 1994. This patient was put in a “watchful position”, and took two herb mixtures “Detoxitea” and “Essiac”. Six years later (July, 2000), the serum PSA level was reported to be 6.4. Then this patient added some natural supplements including Epilobium and liquid selenium. The serum PSA level has been monitored regularly since that time as displayed in following table.

TABLE 6
Serum PSA level

| Time | Serum PSA (μg/L) | Ref. Range (μg/L) |
|---|---|---|
| 5 Jul. 1994 | 4.4 | 0.0-4.0 |
| 17 Jul. 2000 | 6.4 | * |
| 15 Nov. 2000 | 7.7 | * |
| 17 Jan. 2001 | 7.6 | * |
| 12 Jun. 2001 | 8.8 | * |
| 17 Sep. 2001 | 10.9 | * |
| 17 Oct. 2001 | 11.2 | * |
| 17 Nov. 2001 | 11.5 | * |
| 30 Nov. 2001 | Start Herb Formula | |
| 15 Jan. 2002 | 10.6 | * |

From the end of November 2001, the patient started therapy by oral administration of the herbal formula of the present invention. The intake dosage of the herbal formula was 2760 mg per day. This herbal treatment continued for one and half months, a follow-up serum PSA level was 10.6. During the two months, the patient's energy and well-being were enhanced, and no adverse effects were reported.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the disclosure.

### BIBLIOGRAPHY

Carilla, E., Briley, M., Fauran, F. Sultan, C. and Duvilliers, C. (1984). Binding of Serenoa repens (Permixon), a new treatment for prostatic benign hyperplasia, to the cytosolic andrgoen receptor in the rat prostate. J Steroid Biochem., 20, 521-523.
Darzynkiewicz, Z., Traganos, F., Wu, J. M. and Chen, S. (2000). Chinese herbal mixture PC SPES in treatment of prostate cancer, Int. J. Oncol., 17, 729-736.
Delos, S., Carsol, J. L., Ghazarossian, E., Raynaud JP., and Martin, P-M. (1995). Testosterone metabolism in primary cultures of human prostate epithelial cells and fibroblasts. J Steroid Biochem. MolMolec. Biol., 55, 375-383.
Iehlé, C., Delos, S., Guirou, O., Tate, R., Raynaud, JP. and Martin, P-M. (1995). Human prostatic steroid 5a-reductase isoforms—a comparative study of selective inhibitors. J. Steroid Biochem. Molec. Biol., 54, 273-79.
Kameda et. al. (1999). Annual meeting of the America Society of Clinical Oncologists, Abstract 1230.
Oh et. al. (2000). Annual meeting of the America Society of Clinical Oncologists, Abstract 1334.
Pfeifer, B. L., Pirani, J. F., Hamann, S. R. and Klippel, K. F. (2000). PC-SPES, A dietary supplement in the treatment of hormone-refractory prostate cancer, BJU Int, 85, 481-485.
Small et. al, (2000). Annual meeting of the America Society of Clinical Oncologists, Abstract 1316.
Voet D, Voet J. G. & Pratt, C. W. (1999). “Fundamentals of Biochemistry”, Eds Wiley & Sons, New York, p 915.

1. An herbal composition comprising Saw Palmetto, Bromelain, Willow Herb, Grape Seed Complex, Wild Rosella, Liquorice, Passionfruit Seed and Selenium yeast.

2. The herbal composition of claim 1 wherein Saw Palmetto is present in a concentration in a range of 5-90% calculated by weight.

3. The composition of claim 1 wherein the Bromelain is present in a range of between 1.5-21.5% calculated by weight.

4. The herbal composition of claim 1 wherein the Willow Herb is present in a range of between 0.5-14.5% calculated by weight.

5. The herbal composition of claim 1 wherein the grape seed complex is present in a range of 0.75-18.5% calculated by weight.

6. The herbal composition of claim 1 wherein the wild rosella is present in a range of 0.5-20% calculated by weight.

7. The herbal composition of claim 1 wherein the liquorice is present in a range of 0.5-50% calculated by weight.

8. The herbal composition of claim 1 wherein the passionfruit seed is present in a range of 0.5-20% calculated by weight.

9. The herbal composition of claim 1 wherein the selenium yeast is present in a range of 0.5-10% calculated by weight.

10. The herbal composition of claim 1 which is used for the treatment or prophylaxis of cancer in a subject in need thereof.

11. The herbal composition of claim 10 wherein the cancer is prostate cancer.

12. The herbal composition of claim 1 which is used to treat inflammation in a subject in need thereof.

13. The herbal composition of claim 1 which is used to treat prostatic hyperplasia in a subject in need thereof.

14-15. (canceled)

16. The herbal composition of claim 10 wherein the subject is a human.

17-19. (canceled)

20. A method of treatment of a cancer or benign prostatic hyperplasia, said method comprising the step of administering to a subject in need of the treatment an effective amount of a composition comprising Saw palmetto, Bromelain, Willow herb, Grape seed complex, Wild rosella, Liquorice, Passionfruit seed and Selenium yeast.

21. The method of claim 20, wherein the composition comprises Saw Palmetto 5-90%; Bromelain 1.5-21.5%; Willow Herb 0.5-14.5%; Grape Seed Complex 0.75-18.5%; Wild Rosella 0.5-20%; Liquorice 0.5-50%; Passionfruit Seed 0.5-20%; and Selenium Yeast 0.5-10% calculated by weight.

22. The method of claim 20 wherein the cancer is prostate cancer.

23. The method of claim 20 wherein prostate health is promoted.

24. The method of claim 20 wherein the subject is human.
